(19) United States
(12) Patent Application Publication
(10) Pub. No.: US 2026/0062369 A1
(43) Pub. Date: Mar. 5, 2026

(54) PROCESS FOR PRODUCING METHANOL

(71) Applicant: L'Air Liquide, Société Anonyme pour l'Etude et l'Eploitation des Procédés Georges Claude, Paris (FR)

(72) Inventors: Lutz JANKO, Frankfurt am Main (DE); Vincent PEÑA, Frankfurt am Main (DE); Nina BLICKHAN, Frankfurt am Main (DE)

(21) Appl. No.: 19/302,957

(22) Filed: Aug. 18, 2025

(30) Foreign Application Priority Data
Aug. 30, 2024 (EP) 24197653

(51) Int. Cl. C07C 29/151; C07C 29/76

(52) U.S. Cl. CPC C07C 29/1518; C07C 29/76

(57) ABSTRACT

A process for producing methanol from a hydrogen-rich and a carbon dioxide-rich reactant gas stream. According to the invention a decompression gas stream obtained in a low-pressure separator and containing hydrogen and carbon dioxide is recycled to one of the two reactant gas streams or the decompression gas stream is divided over two substreams and the respective substreams are recycled to the respective reactant gas streams. Combination of these streams is carried out before compression to synthesis pressure and before formation of the actual synthesis gas stream.

### CROSS REFERENCE TO RELATED APPLICATIONS

This application claims the benefit of priority under 35 U.S.C. § 119 (a) and (b) to European Patent Application No. EP24187653, filed Aug. 30, 2024, the entire contents of which are incorporated herein by reference.

### BACKGROUND

The invention relates to a process for producing methanol from a first hydrogen-rich and a second carbon dioxide-rich reactant gas stream and from a hydrogen- and carbon dioxide-containing decompression gas stream.

Methanol is produced on a large industrial scale by reaction of synthesis gas over a suitable solid catalyst at synthesis pressures of up to 100 bar. Synthesis gas is typically a mixture of predominantly hydrogen (H₂), carbon monoxide (CO) and carbon dioxide (CO₂). The term “carbon oxides” is often used to encompass carbon monoxide and carbon dioxide. Predominantly the following equilibrium reactions (1) and (2) occur simultaneously over a solid methanol synthesis catalyst.

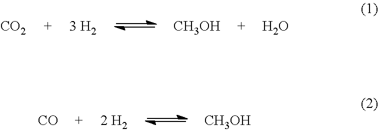

Methanol may also be produced from synthesis gas which is low in, or even free from, carbon monoxide. In this case it is predominantly reaction equation (1) that applies in the methanol synthesis. Production of this so-called carbon dioxide-based methanol has a role especially in processes which are to be realized without, or at least with the lowest possible amount, of fossil energy carriers or fossil inputs and which are intended to generate the lowest possible greenhouse gas emissions.

Thus, carbon dioxide-based methanol is producible for example on the basis of carbon dioxide generated by combustion of a fossil input and renewably produced hydrogen. The renewably produced hydrogen is preferably produced by electrolysis of water on the basis of renewably produced electricity.

A cascade of at least two serially arranged separators is typically arranged downstream of the actual methanol synthesis reactor. In a first high-pressure separator crude methanol is separated as a liquid and at least a portion of the remaining gaseous phase containing unreacted synthesis gas is recycled to the inlet of the methanol synthesis reactor. The raw methanol discharged from the high-pressure separator is introduced into a low-pressure separator after pressure reduction. The pressure reduction causes further unconverted synthesis gas to be outgassed from the raw methanol. This mixture known as decompression gas is typically incinerated or sent for use as fuel. The amount of this expansion gas is so small and the pressure thereof so low that in most cases it is uneconomic to recycle it to the synthesis gas input stream. Since the synthesis gas input stream typically has a higher pressure than the decompression gas stream discharged from the low-pressure separator, the decompression gas stream would initially require compression by a dedicated compressor to allow combination thereof with the synthesis gas input stream.

In carbon dioxide-based methanol synthesis, the synthesis gas input stream often has a lower pressure than the synthesis gas input stream of a conventional methanol synthesis. WO 2021/110565 therefore proposes mixing the decompression gas stream with the synthesis gas make-up gas stream and subsequently compressing the mixture to synthesis pressure to send the decompression gas stream to a use associated with lower carbon dioxide emissions.

Carbon dioxide-based methanol synthesis typically comprises providing two reactant gas streams, thus distinguishing this type of synthesis from a conventional methanol synthesis. Conventional methanol synthesis typically comprises compressing a single synthesis gas input stream which is discharged from a reformer or gasifier for example to synthesis pressure and converting said stream into methanol. By contrast, in carbon dioxide-based methanol synthesis the two reactant gas streams, the hydrogen-rich and the carbon dioxide-rich reactant gas stream, often have different pressures. These two streams are therefore often compressed to synthesis pressure separately from one another and only subsequently combined for the conversion in the methanol synthesis reactor. A mixing of the decompression gas stream with the compressed synthesis gas stream is then either impossible or economically and technologically infeasible since a dedicated compressor would be required for the relatively small decompression gas stream.

### SUMMARY

It is accordingly an object of the present invention to propose a process which at least partially overcomes the aforementioned disadvantage.

One aspect of the invention provides a process for producing methanol, wherein the process comprises the following process steps:
(a) providing a first reactant gas stream rich in hydrogen (H₂);
(b) providing a second reactant gas stream rich in carbon dioxide (CO₂);
(c) providing a decompression gas stream containing hydrogen and carbon dioxide;
(d1) combining the decompression gas stream with the first reactant gas stream before compression to synthesis pressure to form a first mixed gas stream and forming a synthesis gas stream by combining the first mixed gas stream with the second reactant gas stream or
(d2) combining the decompression gas stream with the second reactant gas stream before compression to synthesis pressure to form a second mixed gas stream and forming a synthesis gas stream by combining the first reactant gas stream with the second mixed gas stream or
(d3) dividing the decompression gas stream into a first substream and a second substream and combining the first substream with the first reactant gas stream before compression to synthesis pressure to form a third mixed gas stream and combining the second substream with the second reactant gas stream before compression to synthesis pressure to form a fourth mixed gas stream and forming a synthesis gas stream by combining the third mixed gas stream with the fourth mixed gas stream;
(e) reacting the synthesis gas stream at synthesis pressure in a reaction apparatus over a solid methanol synthesis catalyst to afford a methanol-containing product gas stream;
(f) separating raw methanol from the product gas stream in a first separator to obtain a first raw methanol stream as a liquid phase and a stream of unreacted synthesis gas as a gaseous phase, wherein at least a portion of the stream of unreacted synthesis gas is recycled to an inlet of the reaction apparatus and the first raw methanol stream is discharged from the first separator;
(g) introducing the first raw methanol stream into a second separator, wherein the pressure in the second separator is reduced relative to the pressure in the first separator, to obtain in the second separator a second raw methanol stream as a liquid phase and the decompression gas stream as a gaseous phase, wherein the second raw methanol stream and the decompression gas stream are discharged from the second separator and wherein the decompression gas stream is provided by the discharging from the second separator.

In the context of the process according to the invention the decompression gas stream is combined with the first hydrogen-rich reactant gas stream or combined with the second carbon dioxide-rich reactant gas stream. Alternatively, the decompression gas stream is divided into two substreams and the respective substream is combined with the hydrogen-rich reactant gas stream and with the second carbon dioxide-rich reactant gas stream. The combining of the respective streams is carried out before compression to synthesis pressure. The actual synthesis gas stream is then formed by combining the respective streams of the actual synthesis gas stream.

In any case the compressing to synthesis pressure according to feature (e) is carried out only once the respective reactant gas stream or the reactant gas streams has/have been combined with the decompression gas stream. In the present context the term “synthesis pressure” is to be understood as meaning that the synthesis gas is compressed to a pressure suitable for methanol synthesis. The compressing may be carried out before or after the combining of the corresponding streams (reactant gas streams, mixed gas streams) to afford the synthesis gas stream.

In one example the first reactant gas stream is formed by electrolysis of water or another suitable reactant.

In a further example the second reactant gas stream is derived from a carbon dioxide separation plant, wherein the carbon dioxide is initially bound by absorption and/or adsorption and subsequently released again by desorption in this separation plant. Alternatively the carbon dioxide in the separation plant may be obtained by cryogenic liquefaction of carbon dioxide optionally followed by distillation.

The reaction apparatus may comprise a plurality of parallel or serially arranged reactors comprising a methanol synthesis catalyst. What is decisive here is merely that the methanol synthesis affords a methanol-containing product gas stream from which a liquid raw methanol stream is obtained by appropriate technical measures such as cooling, condensation and separation. This liquid raw methanol stream is initially supplied to a first separator also referred to as a high-pressure separator.

The term “raw methanol” is to be understood as meaning a mixture containing at least methanol (CH₃OH), water and unavoidable impurities such as higher alcohols and ketones.

The conversion of the synthesis gas into methanol and water in the reaction apparatus is carried out at synthesis pressure, for example a pressure of 40 bar to 100 bar, preferably 75 bar to 90 bar. Typical pressure ranges used in the methanol synthesis are well known to those skilled in the art.

The first separator or high-pressure separator effects a gas-liquid separation to obtain a stream of unreacted synthesis gas and a first raw methanol stream. The stream of unreacted synthesis gas may also be referred to as a recycle stream or recycle gas stream. This stream is recycled to an inlet of the reaction apparatus. If the reaction apparatus comprises a plurality of reactors, the term inlet may refer to any inlet of a reactor. What is decisive here is merely that the unreacted synthesis gas is again subjected to reaction at the methanol synthesis catalyst.

The second separator or low-pressure separator effects a further gas-liquid separation. The pressure in the second separator is reduced compared to the first separator. The pressure of the raw methanol flow supplied to the second separator may be reduced by a pressure reducing valve for example. As a result of the pressure reduction, the decompression gas stream is obtained in the second separator. Said stream is recycled to the first reactant gas stream or recycled to the second reactant gas stream or divided into two substreams which are respectively recycled to the first reactant gas stream and the second reactant gas stream. The decompression gas stream is thus not recycled to a previously premixed synthesis gas stream formed from first and second reactant gas stream.

This allows the process to be made more flexible. On the basis of this it is especially possible to decide whether the two main reactant gas streams (the hydrogen-rich and the carbon dioxide-rich stream) will be compressed separately from one another or whether these will be compressed together. In the first case the use of the decompression gas stream as a portion of the input gas stream is only possible if the latter is not supplied to the previously premixed synthesis gas stream. In case of separate compression the pressure downstream of the compressor after combining the streams is too high to be able to supply the decompression gas stream at this point.

A preferred embodiment of the process according to the invention is characterized in that
(h1) the first mixed gas stream and the second reactant gas stream are compressed to synthesis pressure separately from one another and combined to form the synthesis gas stream after compression to synthesis pressure or
(h2) in that the first reactant gas stream and the second mixed gas stream are compressed to synthesis pressure separately from one another and combined to form the synthesis gas stream after compression to synthesis pressure or
(h3) in that the third mixed gas stream and the fourth mixed gas stream are compressed to synthesis pressure separately from one another and combined to form the synthesis gas stream after compression to synthesis pressure.

A separate compression of the two main reactant gas streams is especially advantageous if both streams are provided at different pressures. At least a precompression of one of the two streams is then often required or different compressors or a plurality of serially arranged compressors per reactant gas side are required.

Separate intermediate storage of both main reactant gas streams is often also required. The intermediate storage of hydrogen is necessary for example if this hydrogen is produced by electrolysis with electricity from a renewable energy source. Shared storage is generally impossible due to the possibility of condensation of carbon dioxide. In this case a separate compression of both streams is therefore required, for example to enable a certain minimum pressure in the respective storage tank or to be able to operate the storage tank in a greater pressure range and accordingly render a greater amount of stored gas usable.

The alternatives of process steps h1 to h3 are correspondingly to be assigned according to the description of the alternatives of process steps d1 to d3. That is to say d1 is combinable with h1, d2 is combinable with h2 and d3 is combinable with h3.

A further alternative embodiment of the process according to the invention is characterized in that
(i1) the first mixed gas stream and the second reactant gas stream are combined to form the synthesis gas stream and the resulting synthesis gas stream is subsequently compressed to synthesis pressure or
(i2) in that the first reactant gas stream and the second mixed gas stream are combined to form the synthesis gas stream, and the resulting synthesis gas stream is subsequently compressed to synthesis pressure or
(i3) in that the third mixed gas stream and the fourth mixed gas stream are combined to form the synthesis gas stream, and the resulting synthesis gas stream is subsequently compressed to synthesis pressure.

The alternatives of process steps i1 to i3 are correspondingly to be assigned according to the description of the alternatives of process steps d1 to d3. That is to say d1 is combinable with i1, d2 is combinable with i2 and d3 is combinable with i3.

A further preferred embodiment of the process according to the invention is characterized in that the first reactant gas stream and the second reactant gas stream have different pressures and in that the decompression gas stream is combined with the reactant gas stream having the lower pressure relative to the other reactant gas stream.

As has already been mentioned above, the first reactant gas stream and the second reactant gas stream generally have different pressures depending on the type of the production source used. It is advantageous to supply the decompression gas stream to that reactant gas stream having the lower pressure before compression to synthesis pressure. The lower the pressure of the reactant gas stream, the lower also may be the pressure in the second separator. The lower the pressure in the second separator, the greater the amount of decompression gas which outgasses from the second raw methanol stream in the second separator and may be utilized for the methanol synthesis after recycling to at least one of the reactant gas streams.

In this embodiment it is further preferable that the decompression gas stream has a higher pressure than the reactant gas stream having the lower pressure relative to the other reactant gas stream, and that the decompression gas stream is not compressed before combination with the reactant gas stream having a lower pressure relative to the other reactant gas stream.

In the two aforementioned preferred embodiments the decompression gas stream is not divided into a first substream and a second substream and no third and fourth mixed gas stream is formed.

A preferred embodiment of the process according to the invention is characterized in that the second reactant gas stream, the second mixed gas stream or the fourth mixed gas stream is subjected to a purification process, especially a purification process for removal of sulfur-containing compounds or for removal of oxygen, before compression to synthesis pressure.

This removes substances which may represent catalyst poisons for the methanol synthesis catalyst or are otherwise disruptive from the aforementioned streams.

It is further preferable that the hydrogen present in the decompression gas is used for reduction of impurities present in the second reactant gas stream.

By way of example the hydrogen present in the decompression gas may be used for hydrogenation of sulfur compounds present in the second reactant gas stream. In this case the second mixed gas stream or the fourth mixed gas stream is accordingly supplied to the corresponding purification process and the hydrogen present in the decompression gas stream is used for hydrogenating sulfur compounds, for example in the context of a hydrodesulfurization process in which sulfur in compound form is ultimately liberated in the form of hydrogen sulfide (H₂S) and the latter is then separated from the stream to be purified by adsorptive methods. This makes it possible to reduce the amount of hydrogen to be imported or reactant hydrogen to be used or even obviate the need for any additional hydrogen.

In a further example the hydrogen present in the decompression gas may be used for catalytic conversion of oxygen into water. The water may then be removed from the respective carbon dioxide-rich stream by adsorption on a suitable adsorbent (for example a molecular sieve).

A preferred embodiment of the process according to the invention is characterized in that
the first reactant gas stream contains at least 50 mol % of hydrogen, preferably at least 70 mol % of hydrogen, more preferably at least 80 mol % of hydrogen, more preferably at least 90 mol % of hydrogen, more preferably at least 95 mol % of hydrogen, more preferably at least 99 mol % of hydrogen, and
the second reactant gas stream contains at least 50 mol % of carbon dioxide, preferably at least 70 mol % of carbon dioxide, more preferably at least 80 mol % of carbon dioxide, more preferably at least 90 mol % of carbon dioxide, more preferably at least 95 mol % of carbon dioxide, more preferably at least 99 mol % of carbon dioxide.

Further constituents present in the first and/or second reactant gas stream may be for example constituents inert under the conditions of the methanol synthesis. The proportion of inert constituents, for example nitrogen, may be particularly high especially in carbon dioxide-rich offgases from industrial processes.

In a further embodiment the first reactant gas stream is a pure hydrogen stream and the second reactant gas stream is a pure carbon dioxide stream.

Neither the first nor the second reactant gas stream are to be regarded as a synthesis gas stream, even after supply of the decompression gas stream. This means that none of the aforementioned streams (first reactant gas stream, second reactant gas stream, first to fourth mixed gas stream) is suitable as such for a methanol synthesis. A (synthesis gas) stream suitable for methanol synthesis is obtained only by combining the corresponding streams.

A further preferred embodiment of the process according to the invention is characterized in that
the first reactant gas stream has a pressure of 1 bar to 90 bar, preferably a pressure of 1 to 50 bar, more preferably a pressure of 1 to 40 bar, and
the second reactant gas stream has a pressure of 1 bar to 90 bar, preferably a pressure of 1 bar to 50 bar, more preferably a pressure of 1 to 40 bar.

A further preferred embodiment of the process according to the invention is characterized in that the first separator has a vessel internal pressure of 40 bar to 100 bar, preferably a vessel internal pressure of 75 bar to 90 bar.

A further preferred embodiment of the process according to the invention is characterized in that the second separator has a vessel internal pressure of 1 bar to 50 bar, preferably a vessel internal pressure of 5 bar to 35 bar.

A further preferred embodiment of the process according to the invention is characterized in that the decompression gas stream discharged from the second separator according to step (g) is subjected to a gas enrichment process to obtain a decompression gas stream in which the hydrogen concentration and/or the carbon dioxide concentration is elevated relative to the decompression gas stream discharged from the second separator, wherein the thus obtained decompression gas stream enriched in hydrogen and/or carbon dioxide is provided as a decompression gas stream according to step (c).

Examples of suitable gas enrichment processes are the separation of the gas components by pressure swing adsorption (PSA) or membrane separation processes or a combination thereof.

A further preferred embodiment of the process according to the invention is characterized in that a substream is diverted from the stream of the unreacted synthesis gas as a purge gas stream, and the purge gas stream is supplied to a hydrogen recovery unit to produce a hydrogen-rich stream, wherein at least one stream from the group of first reactant gas stream, second reactant gas stream, first mixed gas stream, second mixed gas stream, third mixed gas stream, fourth mixed gas stream and decompression gas stream is enriched in hydrogen by supplying hydrogen from the hydrogen-rich stream before compression to synthesis pressure.

Suitable hydrogen recovery units contain for example apparatuses for separating gas components by pressure swing adsorption (PSA) or membrane separation processes or a combination thereof.

The hydrogen-rich stream preferably contains at least 70 mol % of hydrogen, more preferably at least 80 mol % of hydrogen, more preferably at least 90 mol % of hydrogen, more preferably at least 95 mol % of hydrogen, more preferably at least 99 mol % of hydrogen.

### BRIEF DESCRIPTION OF THE DRAWING

The invention is more particularly elucidated hereinbelow by way of an exemplary embodiment without in any way limiting the subject matter of the invention. In the FIGURES:

FIG. 1 shows a greatly simplified process flow diagram of an example of the process according to the invention for producing methanol.

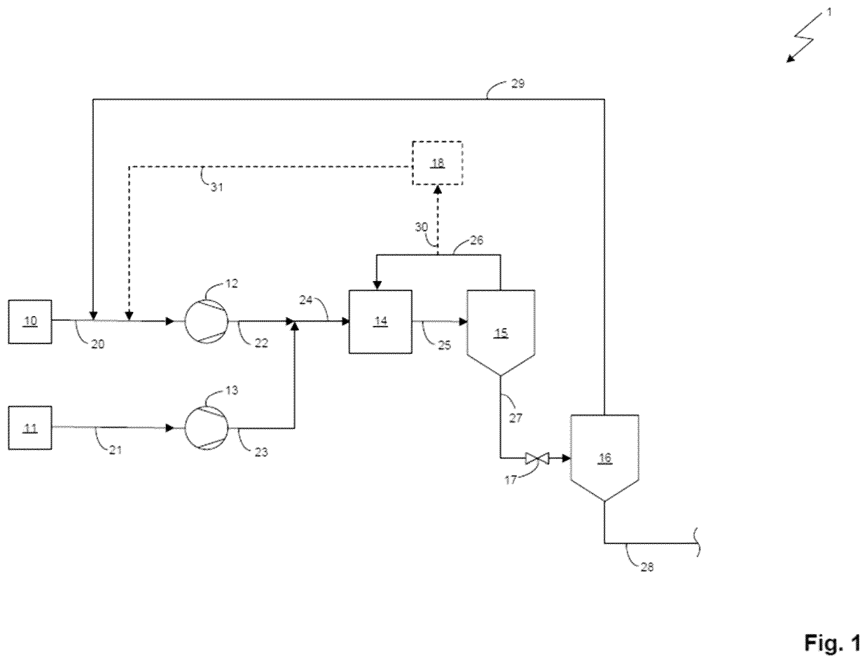

### DETAILED DESCRIPTION OF PREFERRED EMBODIMENTS

A first hydrogen-rich reactant gas stream is provided from a hydrogen source 10, for example an electrolysis plant. The first reactant gas stream may have a hydrogen content of more than 99% by weight for example.

A second carbon dioxide-rich reactant gas stream is provided from a carbon dioxide source 11, for example a carbon dioxide separation plant. The second reactant gas stream may have a carbon dioxide content of more than 99% by weight for example.

The first reactant gas stream is passed on via conduit 20 and combined with a decompression gas stream from conduit 29. This forms a first mixed gas stream. The decompression gas stream is composed substantially of hydrogen and carbon dioxide. The first mixed gas stream is optionally enriched in hydrogen with a hydrogen-rich stream from conduit 31. The hydrogen-rich stream in conduit 31 is produced in an apparatus for hydrogen recovery 31. Alternatively, the hydrogen-rich stream from conduit 31 may be mixed with the first reactant gas stream and the hydrogen-enriched first reactant gas stream is subsequently mixed with the decompression gas stream from conduit 29 to obtain a hydrogen-enriched first mixed gas stream.

The resulting first mixed gas stream is passed on via conduit 20 and compressed to a synthesis pressure suitable for methanol synthesis in a compressor 12.

The pressure in conduit 29 is higher than the pressure in conduit 20. Accordingly the introduction of the decompression gas stream from conduit 29 into conduit 20 does not require a compressor arranged in conduit 29.

The second reactant gas stream is passed on via conduit 21 and compressed to a pressure suitable for methanol synthesis in compressor 13. The pressure in conduit 21 is higher than in conduit 20. The decompression gas stream is therefore introduced into conduit 20 from a second separator 16 via conduit 29. The lower pressure in conduit 20 allows the pressure in the second separator 16 configured as a low-pressure separator to be lower so that a greatest possible amount of decompression gas is obtained from the second separator 16.

The compressed first mixed gas stream is passed on via conduit 22 and the compressed second reactant gas stream is passed on via conduit 23. Both of the aforementioned streams are then combined in conduit 24 to obtain a synthesis gas stream suitable for methanol synthesis in conduit 24.

Due to the separate compression of the streams in conduit 20 and 21 a recycling of the decompression gas stream from conduit 29 to the already mixed synthesis gas stream is not possible. According to the invention the decompression gas stream from conduit 29 is therefore supplied to the as yet uncompressed first reactant gas stream in conduit 20 to form the first mixed gas stream.

The synthesis gas stream compressed to synthesis pressure is introduced via conduit 24 into a reaction apparatus 14 which comprises at least one methanol synthesis reactor having a suitable fixed bed comprising a methanol synthesis catalyst. The methanol synthesis catalyst may be any suitable, for example copper-based, catalyst known to those skilled in the art. In reaction apparatus 14 the synthesis gas stream is reacted in an exothermic equilibrium reaction to afford a methanol-containing product gas mixture which contains methanol and water and unavoidable byproducts. The resulting product gas stream is then cooled below the dew point of methanol or water in order to condense primarily methanol, water and condensable byproducts as a raw methanol stream. The apparatuses necessary therefor (cooler, condenser) are not shown. The biphasic stream which contains not only the condensed constituents but also unconverted synthesis gas is introduced via a conduit 25 into a first separator 15 which is configured as a high-pressure separator.

In the first separator 15 the liquid phase is separated from the gaseous phase. The gaseous phase is composed largely of synthesis gas unconverted in the reaction apparatus 14 which is discharged from the first separator via conduit 26 and after renewed compression to synthesis pressure is recycled using a compressor (not shown) arranged within the conduit 26 to an inlet (not shown) of the reaction apparatus 14 for further conversion into methanol. The gas mixture in conduit 26 is also referred to as recycle gas. A portion of this recycle gas is diverted as purge gas via conduit 30 to prevent enrichment of inert constituents in the methanol synthesis circuit. The purge gas in conduit 30 is supplied to the apparatus for hydrogen recovery 18 to obtain a hydrogen-enriched stream from the purge gas. The apparatus for hydrogen recovery may be for example an apparatus which produces a pure hydrogen stream by the principle of pressure swing adsorption (PSA) or a membrane which produces a hydrogen-enriched carbon dioxide-containing stream.

Also discharged from the first separator 15 via conduit 27 is a first raw methanol stream which is decompressed via a pressure reduction valve 17 and subsequently introduced into the second separator 16. The pressure in the second separator 16 is selected such that it is slightly higher than the pressure in the conduit 20 conducting the first reactant gas stream.

A second raw methanol stream is also discharged from the second separator 16 via conduit 28. This raw methanol stream is supplied to a further workup to obtain pure methanol, for example a thermal separation apparatus (distillation) (not shown).

The decompression gas stream contains about 0.4% of the hydrogen amount supplied via the first reactant gas stream. The decompression gas stream also contains about 1.1% of the carbon dioxide amount supplied via the second reactant gas stream. Accordingly the consumption of hydrogen and carbon dioxide reactant is reduced, thus resulting in a corresponding reduction in the carbon dioxide emissions and in a lower total energy demand for producing the hydrogen by electrolysis.

### LIST OF REFERENCE NUMERALS

1 Process according to the invention
10 Hydrogen source
11 Carbon dioxide source
12, 13 Compressor
14 Reaction apparatus
15 First separator (high-pressure separator)
16 Second separator (low-pressure separator)
17 Pressure reducing valve
18 Apparatus for hydrogen recovery
20-31 Conduit

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A process for producing methanol, wherein the process comprises the following process steps:
(a) providing a first reactant gas stream rich in hydrogen;
(b) providing a second reactant gas stream rich in carbon dioxide;
(c) providing a decompression gas stream containing hydrogen and carbon dioxide;
(d1) combining the decompression gas stream with the first reactant gas stream before compression to synthesis pressure to form a first mixed gas stream and forming a synthesis gas stream by combining the first mixed gas stream with the second reactant gas stream or
(d2) combining the decompression gas stream with the second reactant gas stream before compression to synthesis pressure to form a second mixed gas stream and forming a synthesis gas stream by combining the first reactant gas stream with the second mixed gas stream or
(d3) dividing the decompression gas stream into a first substream and a second substream and combining the first substream with the first reactant gas stream before compression to synthesis pressure to form a third mixed gas stream and combining the second substream with the second reactant gas stream before compression to synthesis pressure to form a fourth mixed gas stream and forming a synthesis gas stream by combining the third mixed gas stream with the fourth mixed gas stream;
(e) reacting the synthesis gas stream at synthesis pressure in a reaction apparatus over a solid methanol synthesis catalyst to afford a methanol-containing product gas stream;
(f) separating raw methanol from the product gas stream in a first separator to obtain a first raw methanol stream as a liquid phase and a stream of unreacted synthesis gas as a gaseous phase, wherein at least a portion of the stream of unreacted synthesis gas is recycled to an inlet of the reaction apparatus and the first raw methanol stream is discharged from the first separator;
(g) introducing the first raw methanol stream into a second separator, wherein the pressure in the second separator is reduced relative to the pressure in the first separator, to obtain in the second separator a second raw methanol stream as a liquid phase and the decompression gas stream as a gaseous phase, wherein the second raw methanol stream and the decompression gas stream are discharged from the second separator and wherein the decompression gas stream is provided by the discharging from the second separator.

2. The process according to claim 1, wherein:
(h1) the first mixed gas stream and the second reactant gas stream are compressed to synthesis pressure separately from one another and combined to form the synthesis gas stream after compression to synthesis pressure or
(h2) in that the first reactant gas stream and the second mixed gas stream are compressed to synthesis pressure separately from one another and combined to form the synthesis gas stream after compression to synthesis pressure or
(h3) in that the third mixed gas stream and the fourth mixed gas stream are compressed to synthesis pressure separately from one another and combined to form the synthesis gas stream after compression to synthesis pressure.

3. The process according to claim 1, wherein the first reactant gas stream and the second reactant gas stream have different pressures and in that the decompression gas stream is combined with the reactant gas stream having the lower pressure relative to the other reactant gas stream.

4. The process according to claim 3, wherein the decompression gas stream has a higher pressure than the reactant gas stream having the lower pressure relative to the other reactant gas stream and in that the decompression gas stream is not compressed before combination with the reactant gas stream having a lower pressure relative to the other reactant gas stream.

5. The process according to claim 1, wherein the second reactant gas stream, the second mixed gas stream or the fourth mixed gas stream is subjected to a purification process before compression to synthesis pressure.

6. The process according to claim 5, wherein the hydrogen present in the decompression gas is used for reduction of impurities present in the second reactant gas stream.

7. The process according to claim 1, wherein:
the first reactant gas stream contains at least 50 mol % of hydrogen, preferably at least 70 mol % of hydrogen, and
the second reactant gas stream contains at least 50 mol % of carbon dioxide.

8. The process according to claim 1, wherein:
the first reactant gas stream has a pressure of 1 bar to 90 bar, and
the second reactant gas stream has a pressure of 1 bar to 90 bar.

9. The process according to claim 1, wherein the first separator has a vessel internal pressure of 40 bar to 100 bar.

10. The process according to claim 1, wherein the second separator has a vessel internal pressure of 1 bar to 50 bar.

11. The process according to claim 1, wherein the decompression gas stream discharged from the second separator according to step (g) is subjected to a gas enrichment process to obtain a decompression gas stream in which the hydrogen concentration and/or the carbon dioxide concentration is elevated relative to the decompression gas stream discharged from the second separator, wherein the thus obtained decompression gas stream enriched in hydrogen and/or carbon dioxide is provided as a decompression gas stream according to step (c).

12. The process according to claim 1, wherein a substream is diverted from the stream of unreacted synthesis gas as a purge gas stream and the purge gas stream is supplied to a hydrogen recovery unit to produce a hydrogen-rich stream, wherein at least one stream from the group of first reactant gas stream, second reactant gas stream, first mixed gas stream, second mixed gas stream, third mixed gas stream, fourth mixed gas stream and decompression gas stream is enriched in hydrogen by supplying hydrogen from the hydrogen-rich stream before compression to synthesis pressure.
